(19) United States
(12) Patent Application Publication
(10) Pub. No.: US 2005/0079147 A1
(43) Pub. Date: Apr. 14, 2005

(54) Wound healing compositions and uses

(76) Inventors: Bernard Delaey, Zingem (BE); Peter G.M.L. De Waele, Zaffelare (BE)

Correspondence Address: HOWREY SIMON ARNOLD & WHITE, 750 Bering Drive, Houston, TX, 77057-2198, US

(21) Appl. No.: 10/744,704

(22) Filed: Dec. 23, 2003

Related U.S. Application Data
(60) Provisional application No. 60/510,830, filed on Oct. 14, 2003.

(51) Int. Cl. A61K 31/715; A61K 31/74

(52) U.S. Cl. 424/78.08; 514/54

(57) ABSTRACT

The present invention relates to poloxamer compositions and the use thereof for the purpose of treating skin wounds on a human body.

This application claims benefit of Provisional Application Ser. No. 60/510,830 filed Oct. 14, 2003, the entire contents of which are incorporated by reference herein.

### FIELD OF THE INVENTION

The present invention relates to poloxamer compositions and the use thereof for the purpose of treating skin wounds on a human body.

### BACKGROUND OF THE INVENTION

In general, several classification systems for wounds can be considered, each varying by the interests of the speciality dealing with the wound. For instance, burn specialists have historically classified burn wounds according to the degree of injury (e.g. first, second and third degree bums), surgeons categorize wounds by severity of tissue loss, while those treating pressure ulcers have their own classification of wounds by stages. Each classification system is useful in generating a prognosis and plan of intervention for each speciality. In the case of skin ulcers, categories often considered are venous leg ulcers, arterial leg ulcers, diabetic ulcers, and decubitus ulcers. Venous leg ulcers are a major cause of morbidity and their care is costly. They necessitate frequent visits to physicians and by visiting nurses, cause loss of productivity in the young and increased frailty in the elderly and commonly lead to hospitalization for often life-threatening cellulitis. In the case of diabetic ulcers, non-healing may frequently be a cause of limb loss.

Skin ulceration is a major health problem and the number of affected subjects is likely to increase as the population ages. Clinically, skin ulcers may be subdivided in several categories, according to their etiology:
venous leg ulcers: caused by venous insufficiency due to malfunctioning of the valves in the veins of the leg
arterial leg ulcers: caused by poor functioning or occlusion of the leg arteries, for instance in the case of arteriosclerosis
diabetic ulcers: caused by impaired microcirculation and sensitory dysfunction as a result of diabetes
decubitus ulcers (pressure ulcers): caused by impaired blood circulation due to prolonged local pressure, e.g. in patients who are immobilized in a hospital bed for long periods.

The prevalence of skin ulceration in the UK, Sweden, and Australia has been estimated at 1% of the adult population. Between 70 and 90 percent of leg ulcers in the UK are of venous origin. In the US, the number of individuals affected by venous leg ulcers is greater than 600,000 (Falanga et al, Arch Dermatol 134: 293-300, 1998). Despite this variety of classification systems, the major types of wounds generally considered include the following types: surgical, traumatic, bums, pressure ulcers, arterial ulcers, venous ulcers, diabetic ulcers, amputations, proliferative scars and wounds associated with immune deficiency syndrome.

One of the traditional methods for treating skin wounds involved the application of simple cotton-based dressings or non-adherent gauze products, tulle gras dressings, absorbent dressings and wound contact layer dressings. Since the 1970s, these technically unsophisticated products have been supplemented with more advanced occlusive dressings which are able to provide a moist healing environment for the wound (Winter, Nature 193: 293-294, 1962; Nemeth et al., Arch Dermatol 127: 1679-1683, 1991). These products include synthetic films, alginates, foams, hydrocolloids, hydrogels, and collagen dressings. Despite the use of optimal wound care, with application of the several types of biological or synthetic dressings that are available, many skin wounds may persist for many years. Hence, there is a clear and urgent need towards more effective treatment products and methods, which are able to improve the healing rate of skin wounds, particularly chronic skin wounds.

Recently, more innovative wound treatments have become available. These include both tissue-engineered skin substitutes and pharmacological agents. The former can be subdivided into allogeneic and autologous products. Whereas autologous skin substitutes function primarily by replacing damaged skin with new tissue of autologous origin, allogeneic products are believed to function rather by the release of repair-stimulating factors in the wound environment (Phillips, Arch. Dermatol. 134: 344-349, 1998). They are therefore often considered as biologically active wound dressings.

Commercially available examples of such biological dressings include Apligraf™/Graftskin (Novartis, Switzerland) and Dermagraft™ (Smith & Nephew, UK). Apart from these commercially available biological dressings, many clinicians have also used allogeneic or autologous keratinocyte cultures for the treatment of skin wounds.

U.S. Pat. No. 5,866,167; U.S. Pat. No. 6,585,969; U.S. Pat. No. 6,126,935 and EP 0 615 545 disclose preparation and use of non-viable keratinocyte lysates or pellet fractions from keratinocyte cell cultures which are suitable for promoting wound healing. Apart from tissue-based products, many pharmacological agents have also been applied as wound healing compositions.

Such compositions use poloxamers as a pharmaceutical vehicle/gel excipient in several applications. For instance EP 0 551 626; WO 86/0813; U.S. Pat. No. 5,298,260, EP 0 455 396 describe various formulations containing a poloxamer which is used as a delivery vehicle for an active ingredient.

During clinical research related to a wound treatment product, formulated in a poloxamer-containing composition, we found that the poloxamer-containing formulation unexpectedly showed wound healing activity by itself.

Advantages of the composition of the present invention include that it is cheap to produce and may be used on the wound for several days without the need to change dressings. This reduces the cost of treatment and at the same time reduces the risk for additional trauma, as is the case with other dressings which have to be changed daily or even several times per day. When a dressing change is needed the present gel may be easily removed from the wound by gentle irrigation with cold water or saline solution.

### SUMMARY OF THE INVENTION

The present invention relates to a method of treating skin wounds of a mammal comprising administering to the skin wound a composition consisting essentially of poloxamer, buffer and one or more pharmaceutically acceptable excipients. The present invention relates to poloxamer or derivatives thereof for use as a medicament.

The present invention also relates to a poloxamer or derivatives thereof for the preparation of a composition for the treatment of skin wounds. The present invention relates to poloxamer or derivatives thereof for use in a composition for the treatment of skin ulcers.

The skin wounds are chronic skin wounds or acute skin wounds. The chronic wounds are characterized as venous leg ulcers, arterial leg ulcers, diabetic ulcers and decubitus ulcers. The acute wounds are typically surgical wounds, abrasions, incisions, lacerations, first degree bums, second degree bums, third degree bums, traumatic wounds, proliferative scars, amputations, corneal wounds, mucosal wounds, etc.

The present invention also relates to a method for the treatment of skin wounds in humans, consisting essentially of or comprising the step of applying to the skin ulcer a poloxamer or derivative thereof.

The composition of the present invention comprises or consists essentially of a poloxamer and may further comprise or consist of other optional elements for the purpose of maintaining a buffered pH, osmolality and stability in any preferred physical form. Such preferred elements may include buffer, salt, sugar, ionic polysaccharide(s), non-ionic polysaccharide(s), humectant and a diluent to bring the composition to a determined volume.

The medicament or composition of the present invention may be utilized in any preferred physical form such as a gel, a cream, a lotion, an ointment, a suspension, a solution, a lyophilized or dry powder or a biocompatible solid matrix.

### BRIEF DESCRIPTION OF THE DRAWINGS

FIG. 1: Rheology of Compositions 1-6, non-sterilized.

FIG. 2: Rheology of Compositions 1-6, steam sterilized.

FIG. 3: Rheology of Compositions 7-10, non-sterilized.

FIG. 4: Rheology of Compositions 7-10, steam sterilized.

FIG. 5: Rheology of Compositions 11-14, non-sterilized.

FIG. 6: Rheology of Compositions 11-14, stem sterilized.

### DETAILED DESCRIPTION OF THE PREFERRED EMBODIMENTS

Unless defined otherwise in this specification, all technical and scientific terms are used herein according to their conventional definitions as they are commonly used and understood by those of ordinary skill in the art of synthetic chemistry, pharmacology and wound treatment.

The present invention provides a method of treating skin wounds of a mammal comprising administering to the surface of the wounded skin a composition consisting essentially of poloxamer, buffer and one or more pharmaceutically acceptable excipients. In a preferred embodiment, the present invention relates to use of a poloxamer or a pharmaceutically acceptable derivative thereof for the preparation of a composition for the treatment of skin wounds. For purposes of this invention, the term “consisting essentially of” limits the inventive composition to poloxamer, buffer and one or more pharmaceutically acceptable excipients such that the basic wound healing properties of the composition are evident. The term “consisting essentially of” in particular serves to exclude from the scope of the composition other pharmaceutically active ingredients, in particular those active ingredients typically used in wound treatments (e.g. antibiotics, antiseptic agents, analgesics, anti-inflammatory agents, anti-infective agents, proteolytic enzyme wound debriders, etc.).

The term “skin ulcers” is defined as difficult to heal wounds of the skin, possibly also involving deeper laying tissues, and can be subdivided in several categories such as venous leg ulcers, arterial leg ulcers, diabetic ulcers and decubitus ulcers.

Poloxamers are a class of nonionic polyoxyethylene-polyoxypropylene block co-polymers with the general formula HO(C₂H₄O)ₐ(—C₃H₆O)_{b}(C2H4O)ₐH. wherein a ranges from 12-101 and b from 20-56. Poloxamers are available in different grades which vary from liquids to solids. In the medical field it is used as a delivery vehicle/gel excipient in formulations.

The “poloxamer” of the present invention may be selected from a group comprising of poloxamer 124, 180, 188, 237, 338, 407 and mixtures thereof. One such preferred poloxamer is poloxamer 407. The concentration of poloxamer ranges from about 1-99% by weight more preferably 5-75%, most preferably 15-30%. One of the many possible methods to use the poloxamer compositions is by dissolving solid poloxamer granules or powder into water under continuous stirring conditions such that the obtained composition is liquid at cold temperature of about 4° C. and becomes a gel at room temperature or on contact with the human skin.

In a particular embodiment, for the purpose of obtaining liquid compositions, the present invention may use poloxamer at concentrations lower than 15% (w/w), for instance a concentration ranging between I and 15%. The composition of poloxamer may be present in concentrations of 1-5%, 5-10%, or 10-15% by weight respectively. In these cases, the poloxamer concentration generally will not be sufficiently high to generate a gel with thermoreversible viscosity as described above. However, the product can still be applied onto the wound in a liquid form. Alternatively, it may be preferred to add one or more other gel-forming compounds to the formulation, so as to obtain a non-thermoreversible hydrophilic gel. Any suitable gel-forming agents known in the art may be used in this respect, provided they are compatible with application onto a wound surface. Examples of such agents include: microcrystalline cellulose, cellulose derivatives (such as ethyl cellulose, methyl cellulose, carboxymethyl cellulose, hydroxypropylmethyl cellulose, hydroxypropyl cellulose), certain gums (such as acacia gum, tragacanth gum) carrageenans, pectine, carbomers, modified starch, colloidal aluminum silicates, etc.

The “buffer” of the present invention may be selected from any suitable buffer known in the art, examples of buffers which are useful in this regard, include citrate, phosphate or other buffers preferably clinically acceptable buffering agents. For example, a buffer may be added to obtain a pH of between 5 to 9, preferably of 6 to 8, most preferably of about 7.

The “sugar” of the present invention may be a mono- or disaccharide. One such example used in the composition of the present invention is sucrose. Additional examples of such sugars useful for the present invention are glucose, maltose, fructose or mixtures thereof added at concentrations of 1-50% (w/w), more preferably at 1-25% (w/w), most preferably at 1-10% (w/w) respectively. As demonstrated in Example 1, it has been found that a composition containing 20.5% poloxamer 407, 0.65% sodium chloride, 0.89% citrate buffer pH 6.8, 6.5% sucrose, all dissolved in water, provides a gel dressing which results in a substantial reduction of wound area within 2 weeks when applied to chronic venous ulcers.

The “salt” of the present invention is any suitable salt known in the art for adjusting the ionic strength/osmolality of the composition of the present invention. For instance, application on very exudative wounds may require a hypertonic gel while for other types of wounds a hypotonic or isotonic gel may be preferred. A preferred salt to be included is sodium chloride. For example an isotonic gel may be obtained by adding sodium chloride to a concentration of 0.9% w/w and a hypertonic or hypotonic gel may be obtained by adding sodium chloride to higher or lower concentrations than 0.9% w/w, respectively.

An example of an ionic polysaccharide for the purpose of the present invention is Xanthan gum added at concentrations of 0.01-1% (w/w), more preferably at 0.05-0.5% (w/w).

An example of a non-ionic polysaccharide for the purpose of the present invention is maltodextrin added at concentrations of 0.1-10% (w/w), more preferably at 0.5-5% (w/w), most preferably at 0.5-2% (w/w).

In an embodiment of the present invention the composition comprises or consists essentially of the following components: 1-30% by weight poloxamer 407 as a medicament, buffer for maintaining a pH of 5-9, 0.5-5% by weight sodium chloride, 1-50% by weight sucrose and optionally 0.01 to 1% by weight xanthan gum and 0.1-10% by weight maltodextrin.

Addition of these further compounds to the poloxamer formulation may be achieved either by adding these compounds in powdered or granulated form to the cooled liquid poloxamer solution under continuous stirring until complete dissolution, or they may first be dissolved into water or an aqueous solution and subsequently mixed with the poloxamer solution or gel.

A further beneficial property of the gel of this invention is that, unlike many other amorphous hydrophilic gels, it is not conducive to the growth of microbial organisms, even in the absence of specific antimicrobial compounds added to the formulation. This property is highly desirable, since it is well known that patients suffering from for instance skin ulcers often display a hypersensitivity to the antimicrobial compounds present in most commercially available hydrogel dressings.

If so desired, the compositions of the present invention may be further enhanced by including a humectant, in order to prevent excessive loss of water by evaporation. Suitable humectants in this respect include glycerol, propylene glycol, sorbitol, maltitol, lactitol, etc. Furthermore, in order to obtain a composition with enhanced microbiological stability preservatives may be added, such as e.g. methyl-, ethyl-, propyl- or butyl paraben, benzyl alcohol, sorbate, chlorhexidine, etc.

The present invention also provides a process for treating a skin wound to promote healing in a human comprising the step of topically applying to the skin ulcer an effective amount of a poloxamer containing composition, optionally in a pharmaceutically acceptable carrier for topical administration.

A further embodiment of the invention is to provide the composition as a dry product, for instance in the form of a powder, granules, a foam, a sheet, a plug, etc. This dry product can then be applied onto a wound, where it will form a gel by adsorbing wound fluid.

Alternatively, the dry formulation may be hydrated prior to use. In certain circumstances, such a dry formulation may offer several advantages, such as ease of use, better capacity to absorb wound fluid and better stability, resulting in a longer shelf life. Preparation of such a dry composition may be achieved in multiple ways. For instance, the components of the composition may be obtained in a dry form and blended together in a suitable mixing system such as a ball mill or similar equipment capable of forming a uniform powder mixture. Alternatively, a liquid preparation as described above may first be prepared, followed by removing the water from the composition by using a suitable drying process. This may be achieved by different dehydration methods available in the art. For instance, when the composition is dried by spray drying, a free flowing powder will be obtained. Freeze drying, on the other hand, may result in a solid “cake”. Such a process therefore offers interesting possibilities to provide the dry composition in the form of a sheet or plug, by first casting the liquid composition in a suitable mold, followed by freeze-drying. The dimensions of the dried sheet thus obtained may be adapted by varying the dimensions of the mold, but after drying it is also possible to further adjust the dimensions by simply cutting the freeze-dried cake to size, in order to precisely fit the size and form of the site to be treated.

The present invention relates to a method of treating skin ulcer, wherein the size of skin ulcer is reduced by at least 20% in surface area, preferably 30%, more preferably 40% compared to the standard compression therapy within a period of about 24 weeks wherein the said method comprises or consists of the step of applying to the skin ulcer a poloxamer or derivative thereof.

The present invention relates to a method of treating skin ulcer, wherein the incidence of ulcer closure within a period of 24 weeks is at least 20% higher than that obtained with standard compression therapy and involves the step of applying to the said ulcer a composition containing a poloxamer or derivative thereof.

For use on wounds or otherwise damaged skin, it is desirable that the compositions of the present invention are provided in a sterile form. This can be achieved by multiple means known in the art. For instance, the individual components may first be sterilized in powdered or granulated form, followed by aseptically dissolving them into a sterile aqueous medium. In this case, sterilization of the powder or granulate may preferably be performed by an irradiation process, such as gamma or electron beam irradiation. Alternatively, the liquid or semi-liquid composition may be sterilized after all components have been mixed and dissolved in an aqueous medium, for instance by steam sterilization, by irradiation with gamma rays or electron beams or by microfiltration through a filter with a small pore size (e.g., 0.2 μm). By virtue of their thermoreversible properties, the present compositions can be easily microfiltrated when sufficiently cooled to be in the liquid state. This is in contrast to most other hydrophilic gels, which, due to their very high viscosity, cannot be easily sterilized by filtration through a small pore size microfilter. For compositions provided in a powdered, granulated or otherwise solid form, sterilization may be achieved by irradiation of the product, preferably after packaging. This is preferably performed by using electron beam irradiation, as it has been found that this form of sterilization results in minimal damage to the macromolecular compounds of the formulation. The irradiation dose to be used in order to obtain sterility is dependent on the initial bioburden of the composition, but useful irradiation doses in this respect are generally between 10 and 30 kGy, preferably between 15 and 25 kGy.

As indicated before, a useful property of the liquid and semi-liquid compositions of the present invention is that they display a thermoreversible viscosity, with compositions kept at a lower temperature than the sol-gel transition temperature being in the liquid state and compositions kept at a higher temperature forming an amorphous hydrophilic gel. This sol-gel transition temperature can be measured accurately by using one of the techniques known in the art. For instance, a Brookfield rotary viscosimeter or a plate-cone rheometer equipped with a thermoregulator may be used. To this effect, the gel or liquid composition is applied to the measuring device at a certain temperature. Viscosity is measured continuously while varying the temperature of the test composition. Upon reaching the sol-gel transition point, the viscosity of the composition will change rapidly. The observed viscosity of the formulation at different temperatures will depend on the exact composition and the concentration of the various components. The sol-gel transition temperature is also dependent on the concentration of the various components of the composition, but generally will range between 10 and 30° C. It should be realized that the sol-gel transition temperature can be modulated to the desired specifications of each application by varying the nature and concentration of the components.

The compositions described herein may be packaged according to the specific needs one has in mind. For instance, examples of useful packaging formats include vials, syringes, tubes, pouches, dispenser flacons and the like. The package may either be intended for single use or for multiple use. In case of multi-use packages, it is preferable to include a system which prevents microorganisms to enter the package after the first opening, in order to keep the contents sterile for subsequent applications. For liquid or semi-liquid compositions, this may be achieved, for instance, by equipping the container with a valve or pump system which allows only unidirectional flow of the gel or liquid, so as to prevent flowback of possibly contaminated product into the container. Another packaging format, possible by virtue of the thermoreversible properties of the liquid and semi-liquid compositions of this invention, is the use of a container equipped with a spray system. When cooled below the gel transition point, the liquid or semi-liquid compositions according to certain embodiments of the present invention have a viscosity which is sufficiently low to allow nebulization. Once sprayed onto the wound, the composition will form a gel in situ. This packaging format is particularly useful for multiple use since it effectively prevents contamination of the contents of the container. A further possibility is to provide the liquid or semi-liquid composition soaked into a suitably absorbent dressing such as a gauze or foam dressing. The soaked dressing itself may then be further packaged into a poach or peelable blister tray.

The powdered or granulate compositions of this invention may be packaged for instance in sachets, bottles or powder spray devices, possibly equipped with a system that allows measured amounts of the powder to be applied to the site to be treated. When provided in the form of a sheet or plug, the compositions may preferably be packaged in a tray with a peelable lid (for instance a blow-formed tray), which maintains the structural integrity of the sheet during storage and transport.

If so desired, the wound treated with the composition of the present invention may be additionally covered with a secondary dressing. Ideally, this additional dressing should be substantially non-absorptive, to prevent the composition of the present invention to be soaked into the dressing. Such preferred examples of useful dressings in this respect include hydrocolloid dressings, polyurethane film dressings, silicone dressings, or any other suitable dressing found to be compatible with the present gel and the specific wound care requirements of the wound to be treated. If required the wound may be additionally covered with a compression bandage, for instance in the case of venous origin.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Use of a Poloxamer Composition for Treatment of Chronic Venous Skin Ulcers

A composition was prepared containing the components as mentioned in Table 1. The composition was liquid below 15° C. and formed a hydrophilic gel when warmed above 20° C. The composition was sterile-filtered using a 0.2 μm cut-off filter and filled into 10 mL glass vials.

TABLE 1

| Compound | Concentration (% w/w) |
|---|---|
| Poloxamer 407, EP* (BASF, US) | 20.25 |
| NaCl, EP | 0.65 |
| Citrate buffer, pH 7, EP | 0.9 |
| Sucrose, EP | 6.5 |
| Water for injections, EP | Ad 100% |

*EP: European Pharmacopoeia

This composition was clinically evaluated on 6 patients presenting with a chronic venous leg ulcer. The median duration of the leg ulcer at the initiation of the study was 55 weeks (range: 6 to 410 weeks), the size ranged from 2-73 cm² (median: 20 cm²). Patients were treated every 3-4 days by applying 0.2 mL of the composition per cm² of ulcer area, for a maximum of 4 applications. Upon application to the wounds, the composition formed an amorphous hydrophilic gel. The ulcers were additionally covered with a hydrocolloid dressing (Comfeel Ulcus®, Coloplast, Demark; 3 patients) or a non-adherent adsorbent dressing (Mepilex®, Mölnlycke, Sweden, 3 patients). All patients were given compression therapy (Rosidal® K, Lohmann & Rauscher, Germany). At each visit, the wounds were clinically assessed and photographed, the wound edges traced on a cellophane sheet and local pain score was assessed on the VAS scale.

At the end of the study (14 days after the first treatment with the composition), a mean reduction of ulcer size of 44% was observed, with one ulcer being completely closed. No significant changes in VAS pain score were reported. In view of the long chronicity of these skin ulcers (median duration: 55 weeks, despite adequate treatment using standard wound care products), this is a surprising result and underscores the clinically beneficial potential of the composition.

### Example 2

### Use of a Poloxamer Composition for Treatment of Chronic Venous Skin Ulcers

A composition containing the following components was prepared aseptically as described in Table 2.

TABLE 2

| Compound | Concentration (% w/w) |
|---|---|
| NaCl, EP* | 0.68 |
| Citrate buffer, pH 6.8, EP | 0.7 |
| Sucrose, EP | 6.5 |
| Maltodextrin, EP | 1 |
| Xanthan gum, EP | 0.1 |
| Water for injections, EP | Ad 100% |

*EP: European Pharmacopoeia

This formulation was filled into 10 mL glass vials (4 mL/vial) and lyophilized under aseptic conditions, yielding a dried powder. After lyophilization, the vials were closed under vacuum and further subjected to sterilization by electron beam irradiation (dose: 15 kGy). Before use for wound treatment, the freeze-dried composition was reconstituted by dissolving the dried powder in 1 mL of cold (approx. 4° C.) sterile NaCl (1% w/w in water for injections) and adding 3 mL of cold (approx. 4° C.) sterile poloxamer 407 solution (27% w/w in water for injections). The composition was mixed thoroughly by repeatedly transferring the liquid between two Luer lock syringes which were connected with a Luer lock connector (Qosina, US).

This reconstituted composition had the following composition as mentioned in Table 3. This composition was liquid below 15° C. and formed a hydrophilic gel when warmed above 20° C. The clinical efficacy of composition was evaluated in an open label, randomised, multicenter, parallel group clinical study. A total of 31 patients were involved: 15 patients received standard compression therapy (control group) and 16 patients received standard compression therapy in addition to the present composition (medication group). Standard compression therapy was achieved by application of a primary, combined with a short stretch compression bandage (Rosidal® K, Lohmann & Rauscher, Germany). Patients included in the study had hard to heal venous leg ulcers of 1-20 cm² existing for at least 6 weeks and not responding to conventional therapy. The primary endpoint of the study was to evaluate the efficacy of the study medication on complete ulcer healing within 24 weeks.

TABLE 3

| Compound | Concentration (% w/w) |
|---|---|
| Poloxamer 407, EP* (BASF, US) | 20.25 |
| NaCl, EP | 0.7 |
| Citrate buffer, pH 6.8, EP | 0.7 |
| Sucrose, EP | 6.5 |
| Maltodextrin, EP | 1 |
| Xanthan gum, EP | 0.1 |
| Water for injections, EP | Ad 100% |

*EP: European Pharmacopoeia

In order to further assure only hard to heal ulcers were evaluated, the study started with a run-in period during which all ulcers were treated with standard compression therapy for 4 weeks. Only those patients with stable ulcers (i.e. ulcer margin showing decrease or increase by less than 0.75 mm/week) were enrolled for further treatment and evaluation (the baseline time point at which treatment started was defined as week 0).

In the control group, the median ulcer area at week 0 was 5.97 cm² (range 1.7-18.3 cm²) and the median ulcer duration was 1.02 years. In the medication group, the median ulcer area at week 0 was 4.39 cm² (0.7-20.3 cm²) and the median ulcer duration was 1 year.

Patients in the medication group received a maximum of 8 applications of the formulation described in Table 3. Five applications were performed with an interval of one week (i.e. application on week 0, 1, 2, 3, 4), followed by 3 applications with an interval of 2 weeks (i.e. application on week 6, 8, 10). In case the wound was closed before the end of the treatment period, application of the study medication was discontinued but standard compression therapy was continued (in this case, dry gauze was used as the primary dressing). For each application of the study medication, the area of the ulcer was measured and 0.5 mL of the formulation of Table 3 was applied per 2.5 cm² of wound area. Within seconds after application onto the wound, the formulation formed an amorphous semisolid gel which did not run off and stuck to the wound. Thereafter, the wound was covered with a primary dressing (Comfeel Ulcus®, Coloplast, Denmark) and the compression bandage was applied. The study medication was left in place for at least 48 hours. After this 48 hour period and if clinically indicated, the compression bandage and primary dressing were removed and replaced by a different primary dressing (Tegapore®, Smith & Nephew, UK) and the compression bandage was re-applied.

Patients in the control group were treated in the same way using primary dressings and compression bandages, except for application of the formulation of Table 3.

Ulcer size was measured planimetrically in both patient groups on weeks 0, 1, 2, 3, 4, 6, 8, 10, 12, 16, 20 and 24. At the primary evaluation endpoint (24 weeks after treatment initiation), it was observed that in the control group 13% of ulcers were completely closed, whereas in the medication group treated with the formulation of Table 3, complete closure was achieved in 50% of the ulcers (i.e. a difference of 37%). When evaluating the reduction in ulcer size, it was found that in the control group the mean ulcer area was reduced by only 13.6% between week 0 and week 24, whereas in the group treated with the formulation of Table 3 a mean reduction of 69.4% was observed. In view of the fact that this study only involved patients suffering from long-standing ulcers not responding to standard therapy, these effects on closure frequency and ulcer size are very remarkable and provide strong evidence of the potent healing properties of this composition.

### Example 3

### Use of Poloxamer Compositions for Treating Burn Wounds

Poloxamer compositions are prepared containing the components as mentioned in Table 1. The compositions are liquid below 15° C. and formed a hydrophilic gel when warmed above 25° C. The compositions are sterile-filtered using a 0.2 μm cut-off filter and filled into 10 mL glass vials.

TABLE 4

| Compound | Concentration (% w/w) |
|---|---|
| Poloxamer 407, EP* (BASF, US) | 18, 20, 22, 24, or 26% |
| NaCl, EP | 0.65% |
| Citrate buffer, pH 6.8, EP | 0.9% |
| Sucrose, EP | 6.5% |
| Water for injections, EP | Ad 100% |

*EP: European Pharmacopoeia

These compositions are useful for the treatment of bum wounds. For that purpose, a composition from Table 4 is applied to the burn wound at an amount of approximately 0.1 to 0.4 ml/cm² of wound area. The burn is then additionally covered with a non-adherent wound dressing. In the case of third degree burns, the composition may be used in combination with standard split thickness mesh grafts, as known by the person skilled in the art. The wounds are given standard care and monitored at a regular basis. If necessary, the composition is re-applied at regular intervals (depending on the wound status) until the wound is closed.

### Example 4

### Compositions Containing Different Poloxamer 407 Concentrations

A series of compositions was prepared, containing variable concentrations of Poloxamer 407 (ranging from 15% to 27% w/w) and fixed concentrations of sucrose, xanthan gum and citrate buffer. The composition of the different formulations is provided in Table 5.

TABLE 5

| | Concentration (% w/w or mM) | | | | | |
|---|---|---|---|---|---|---|
| | Composition N^{o}: | | | | | |
| Compound: | 1 | 2 | 3 | 4 | 5 | 6 |
| Poloxamer 407, | 15% | 18% | 20% | 22% | 25% | 27% |
| EP (BASF, US) | | | | | | |
| Sucrose, EP | 6.5% | 6.5% | 6.5% | 6.5% | 6.5% | 6.5% |
| XG, EP | 0.1% | 0.1% | 0.1% | 0.1% | 0.1% | 0.1% |
| Citrate buffer, | 30 mM | 30 mM | 30 mM | 30 mM | 30 mM | 30 mM |
| pH 6.8, EP | | | | | | |
| Deionized water | ad 100% | ad 100% | ad 100% | ad 100% | ad 100% | ad 100% |

From all compositions, a 4 g aliquot was filled into 10 mL glass vials and sterilized by autoclaving for 30 minutes at 121° C. Samples from both the non-sterilized and the steam-sterilized compositions were analysed by rheology. This was done by using a TA Instruments AR2000 rheometer equipped with a thermoregulated 4 cm², 2° TA steel cone/plate geometry. All analyses were performed using a oscillation procedure with the following parameters: controlled strain: 0.5%, frequency: 1 Hz, temperature range: 4° C. to 40° C., temperature change rate: 1° C./min.

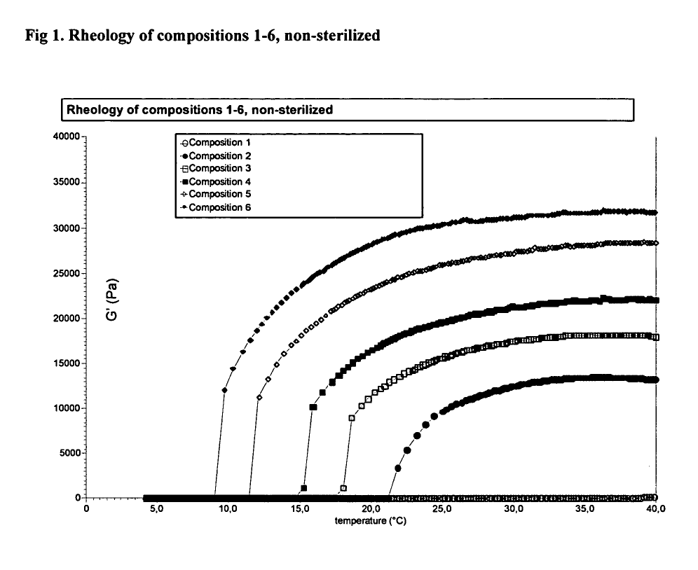

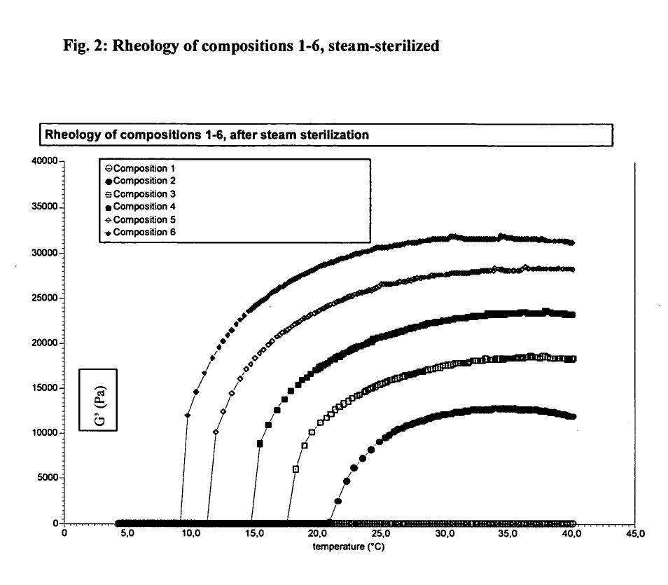

Results from the rheology measurements are represented graphically in FIGS. 1 and 2, showing the storage modulus G′ in function of the temperature. The data are summarised in Table 6: for the purpose of this invention, the gelling temperature was obtained by determining the temperature at which a G′ of 2000 Pa was reached.

TABLE 6

| | Non-sterilized | | | | | | Steam-sterilized | | | | | |
|---|---|---|---|---|---|---|---|---|---|---|---|---|
| | Composition: | | | | | | | | | | | |
| | 1 | 2 | 3 | 4 | 5 | 6 | 1 | 2 | 3 | 4 | 5 | 6 |
| Gelling point | NA* | 21.5 | 18.3 | 15.6 | 11.7 | 9.2 | NA* | 21.6 | 18.0 | 15.0 | 11.6 | 9.2 |
| temperature | | | | | | | | | | | | |
| (° C.) | | | | | | | | | | | | |
| G′ at 37° C. (Pa) | 33 | 13900 | 18600 | 23500 | 28250 | 31250 | 4 | 13500 | 18250 | 22000 | 28100 | 32000 |

*Composition 1 did not form a gel within the measured temperature range

These tests show that, by varying the concentration of Poloxamer 407, it is possible to generate compositions with a variable gelling point temperature and elastic modulus. Also, the data show that it is possible to sterilise such compositions by autoclaving, without significantly altering their rheological characteristics.

### Example 5

### Compositions Containing 25% (w/w) or 27% (w/w) Poloxamer 407, 1% Sucrose, 0.1% Xanthan Gum, 30 mM Citrate Buffer and 0% or 0.1% Sodium Chloride

Poloxamer 407-containing compositions were prepared as provided in Table 7:

TABLE 7

| | Concentration (% w/w or mM) | | | |
|---|---|---|---|---|
| | Composition N^{o}: | | | |
| Compound | 7 | 8 | 9 | 10 |
| Poloxamer 407, EP | 25% | 25% | 27% | 27% |
| (BASF, US) | | | | |
| Sucrose, EP | 1% | 1% | 1% | 1% |
| XG, EP | 0.01% | 0.01% | 0.01% | 0.01% |
| Citrate buffer, pH | 30 mM | 30 mM | 30 mM | 30 mM |
| 6.8, EP | | | | |
| NaCl, EP | 0.1% | 0% | 0.1% | 0% |
| Deionized water | ad 100% | ad 100% | ad 100% | ad 100% |

All compositions formed clear gels when heated above their gel point temperature. From all compositions, a 4 g aliquot was filled into 10 mL glass vials and sterilized by autoclaving for 30 minutes at 121° C.

Samples from both the non-sterilized and the steam-sterilized compositions were analysed by rheology. This was done by using a TA Instruments AR2000 rheometer equipped with a thermoregulated 4 cm², 2° TA steel cone/plate geometry. All analyses were performed using a oscillation procedure with the following parameters: controlled strain:0.5%, frequency: 1 Hz, temperature range: 4° C. to 40° C., temperature change rate: 1° C./min.

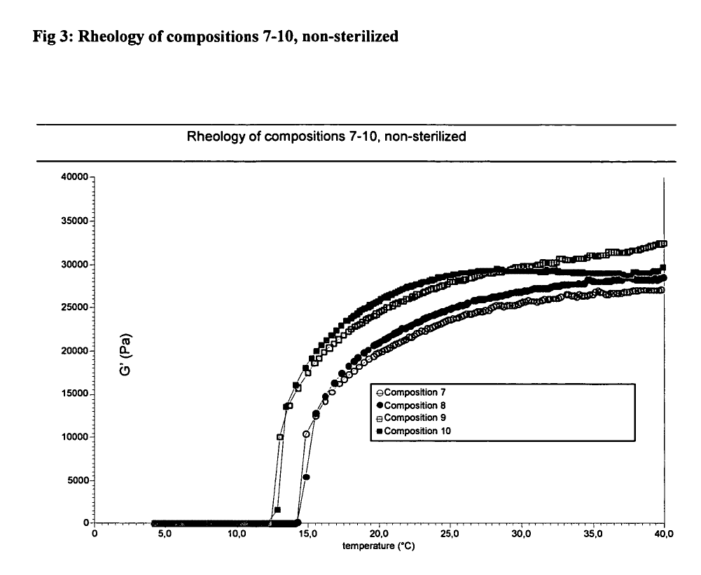

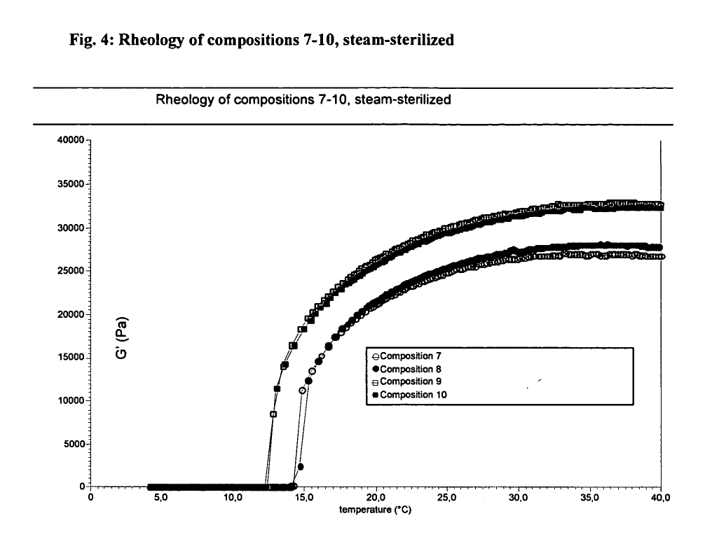

Results from the rheology measurements are represented graphically in FIGS. 3 and 4, showing the storage modulus G′ in function of the temperature. The data are summarised in Table 8: for the purpose of this invention, the gelling temperature was obtained by determining the temperature at which a G′ of 2000 Pa was reached.

When compared with the data from Example 5, these data show that the gelling point temperature may be modified not only by changing the poloxamer concentration, but also by changing the concentration of other additives within the composition. For instance, a steam-sterilized composition containing 25% Poloxamer 407, 30 mM citrate buffer, 6.5% sucrose and 0.1% xanthan gum (composition 5 from Example 4) has a gelling point of 11.6° C., but after reducing the sucrose and xanthan gum concentrations to 1° and 0.01%, respectively (composition 8 from Example 5), the gelling point temperature raises to 14.6° C. Nevertheless, the storage modulus at 37° C. of both compositions is similar (28100 and 28000 Pa for the sterilized compositions 5 and 8, respectively). This property is beneficial because it allows to prepare compositions which form a gel at a certain desired temperature without compromising on the gel strength of the composition when it is placed onto a wound.

TABLE 8

| | Non-sterilized | | | | Sterilized | | | |
|---|---|---|---|---|---|---|---|---|
| | Composition: | | | | | | | |
| | 7 | 8 | 9 | 10 | 7 | 8 | 9 | 10 |
| Gelling point | 14.1 | 14.2 | 12.4 | 12.7 | 14.3 | 14.5 | 12.3 | 12.5 |
| temperature | | | | | | | | |
| (° C.) | | | | | | | | |
| G′ at 37° C. | 27250 | 28500 | 31500 | 29400 | 27000 | 28000 | 32750 | 33750 |
| (Pa) | | | | | | | | |

### Example 6:

### Compositions Containing Different Buffer Systems

Poloxamer 407-containing compositions were prepared as provided in Table 9:

TABLE 9

| | Concentration (% w/w or mM) | | | |
|---|---|---|---|---|
| | Composition N^{o}: | | | |
| Compound | 11 | 12 | 13 | 14 |
| Poloxamer 407, EP | 20% | 20 | 20 | 20 |
| (BASF, US) | | | | |
| Sucrose, EP | 6.5% | 6.5% | 6.5% | 6.5% |
| XG, EP | 0.1% | 0.1% | 0.1% | 0.1% |
| Citrate buffer, pH | 30 mM | 30 mM | 0 mM | 0 mM |
| 6.8, EP | | | | |
| Phosphate buffer, pH | 0 mM | 0 mM | 10 mM | 10 mM |
| 7.6 | | | | |
| NaCl, EP | 0% | 0.7% | 0% | 0.7% |
| Deionized water | ad 100% | ad 100% | ad 100% | ad 100% |

All compositions without NaCl formed clear gels when heated above their gel point temperature, compositions containing 0.7% NaCl formed slightly cloudy gels. From all compositions, a 4 g aliquot was filled into 10 mL glass vials and sterilized by autoclaving for 30 minutes at 121° C.

Samples from both the non-sterilized and the steam-sterilized compositions were analysed by rheology. This was done by using a TA Instruments AR2000 rheometer equipped with a thermoregulated 4 cm², 2° TA steel cone/plate geometry. All analyses were performed using a oscillation procedure with the following parameters: controlled strain:0.5%, frequency: 1 Hz, temperature range: 4° C. to 40° C., temperature change rate: 1° C./min.

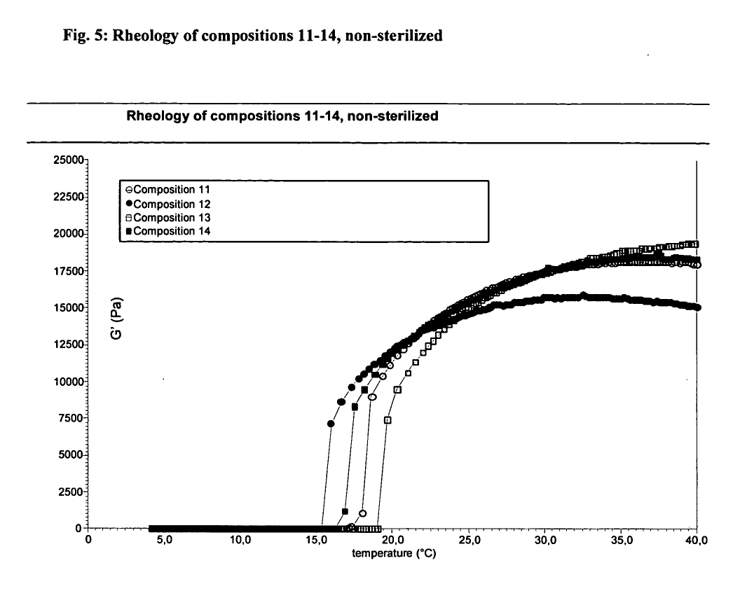

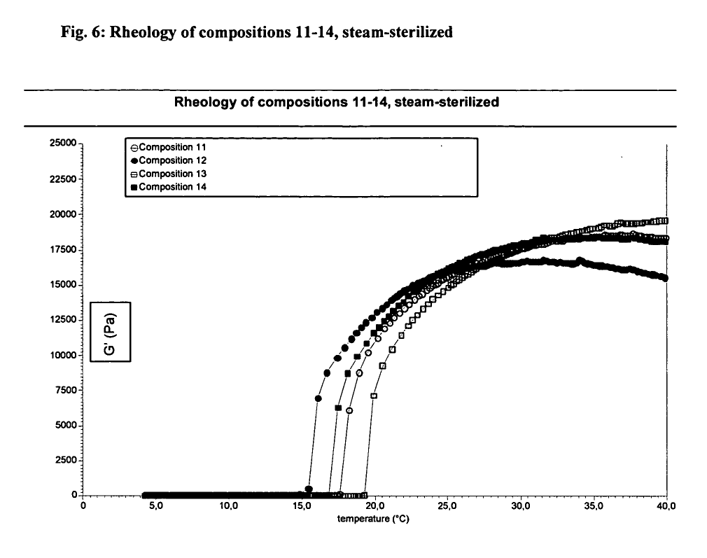

Results from the rheology measurements are represented graphically in FIGS. 5 and 6, showing the storage modulus G′ in function of the temperature. The data are summarised in Table 10: for the purpose of this invention, the gelling temperature was obtained by determining the temperature at which a G′ of 2000 Pa was reached.

TABLE 10

| | Non-sterilized | | | | Sterilized | | | |
|---|---|---|---|---|---|---|---|---|
| | Composition: | | | | | | | |
| | 11 | 12 | 13 | 14 | 11 | 12 | 13 | 14 |
| Gelling point | 17.9 | 15.4 | 19 | 16.9 | 17.7 | 15.6 | 19.4 | 17.1 |
| temperature | | | | | | | | |
| (° C.) | | | | | | | | |
| G′ at 37° C. | 18000 | 15500 | 19000 | 18500 | 18750 | 16500 | 19500 | 18750 |
| (Pa) | | | | | | | | |

This example shows that gel-forming formulations may be prepared with several biologically acceptable buffer systems. Also, it shows that increasing the ionic strength, for example by raising the NaCl or buffer concentration, results in a lowering of the gelling point temperature. When both the NaCl and the buffer concentration are increased, this also results in a lowering of the G′ modulus at 37° C.

### Example 7

### Poloxamer-Containing Compositions With Increased Viscosity at Low Temperatures

Poloxamer 407-containing compositions were prepared as provided in Table 11:

TABLE 11

| | Concentration (% w/w or mM) | | | |
|---|---|---|---|---|
| | Composition N^{o}: | | | |
| Compound | 15 | 16 | 17 | 18 |
| Poloxamer 407, EP | 15% | 15% | 18% | 18% |
| (BASF, US) | | | | |
| Sucrose, EP | 25% | 25% | 25% | 25% |
| XG, EP | 1% | 1% | 1% | 1% |
| Citrate buffer, pH | 30 mM | 30 mM | 0 mM | 0 mM |
| 6.8, EP | | | | |
| NaCl, EP | 5% | 0% | 5% | 0% |
| Deionized water | ad 100% | ad 100% | ad 100% | ad 100% |

From all compositions, a 4 g aliquot was filled into 10 mL glass vials and sterilized by autoclaving for 30 minutes at 121° C.

Composition 15 formed a cloudy, thick-flowing liquid. Composition 16 formed a clear, extremely viscous liquid. Composition 17 formed a cloudy semi-solid paste. Composition 18 formed a clear semi-solid paste. All compositions essentially retained their viscous or paste-like nature, even when cooled down to 4° C. Such compositions are of use in those cases where an outspoken temperature-dependent gelation of viscosity increase is not desired.

1. A method of treating skin wounds of a mammal comprising administering to the skin wound a composition consisting essentially of poloxamer, buffer and one or more pharmaceutically acceptable excipients.

2. The method of claim 1, wherein the skin wounds are chronic skin wounds or acute skin wounds.

3. The method of claim 2, wherein the chronic skin wounds are venous leg ulcers, arterial leg ulcers, diabetic ulcers or decubitus ulcers.

4. The method of claim 2, wherein the acute wounds are surgical wounds, abrasions, incisions, lacerations, first degree burns, second degree burns, or third degree bums.

5. The method of claim 1, wherein the composition is in the form of a gel, a cream, a lotion, an ointment, a suspension, a solution, a lyophilized or dry powder, or a biocompatible solid matrix.

6. The method of claim 1, wherein said poloxamer is selected from the group consisting of poloxamer 124, 180, 188, 237, 338, 407 and mixtures thereof.

7. The method of claim 1, wherein the composition consists of a poloxamer, buffer, salt, sugar; and optionally ionic and/or non-ionic polysaccharide(s) and/or humectant and/or a diluent to bring the composition to a desired volume.

8. The method of claim 1, wherein one of the pharmaceutically acceptable excipients is a salt.

9. The method of claim 1, wherein the pharmaceutically acceptable excipients include sugar, ionic and/or non-ionic polysaccharide(s), a humectant and/or a diluent to bring the composition to a desired volume.

10. The method of claim 1, wherein the composition consists of the following components:
1-30% (w/w) poloxamer;
buffer for maintaining a pH of between about 5 and about 9;
0.5-5% (w/w) sodium chloride;
1-50% (w/w) sucrose;
water;
and optionally
0.01 to 1% (w/w) xanthan gum; or
0.1-10% (w/w) maltodextrin.

11. The method of claim 10, wherein the poloxamer is Polaxamer 407.

12. The method of any one of claims 1, 7 or 10, wherein the buffer is phosphate or citrate.

13. The method of claims 7 or 8, wherein the salt is sodium chloride.

14. The method of claims 7 or 9, wherein the sugar is a mono- or disaccharide.

15. The method of claims 7 or 9, wherein said ionic polysaccharide is xanthan gum and said non-ionic polysaccharide is maltodextrin.

16. The method of claims 7 or 9, wherein the diluent is water.

17. The method of claim 1, wherein between about 0.1 mL to about 1.0 mL of the composition is applied to each cm² wound surface.

18. The method of claim 17, wherein the composition is applied to the wound every 1 to 14 days.

19. The method of claim 17, wherein the composition is applied once every week.
